(19) United States
(12) Patent Application Publication
(10) Pub. No.: US 2010/0196885 A1
(43) Pub. Date: Aug. 5, 2010

(54) METHODS OF VALIDATING CANDIDATE COMPOUNDS FOR USE IN TREATING COPD AND OTHER DISEASES

(76) Inventors: Gerard M. Turino, New York, NY (US); Yong Y. Lin, Bridgewater, NJ (US); Shuren Ma, Cliffside Park, NJ (US); Seymour Lieberman, New York, NY (US)

Correspondence Address: BRYAN CAVE LLP, 1290 AVENUE OF THE AMERICAS, NEW YORK, NY, 10104, US

(21) Appl. No.: 12/449,124

(22) PCT Filed: Jan. 22, 2008
(86) PCT No.: PCT/US08/00871

§ 371 Date: Apr. 19, 2010

Related U.S. Application Data
(60) Provisional application No. 60/881,949, filed on Jan. 22, 2007.

(51) Int. Cl. C12Q 1/68; C12Q 1/00; G01N 33/00; C12Q 1/02

(52) U.S. Cl. 435/6; 435/4; 436/96; 435/29

(57) ABSTRACT

The present invention relates to methods of diagnosing and treating elastin fiber injuries. In additional preferred embodiments, the present invention relates to methods of validating candidate compounds for use in treating chronic obstructive pulmonary disease (COPD), chronic bronchitis, emphysema, refractory asthma, and other related diseases. Examples of such methods include determining if the candidate compound decreases the degradation of elastic fiber in a patient administered the candidate compound by measuring, using mass spectrometry, a marker of elastic fiber degradation in a sample of a body fluid or a tissue of the patient. The invention provides that a decrease in the presence of the marker compared to a control validates that the candidate compound is effective to treat, prevent, or ameliorate the disease.

### FIELD OF THE INVENTION

The present invention relates to the field of elastin fiber injuries and, more particularly, methods of diagnosing and treating elastin fiber injuries. Still further, the present invention relates to methods of validating candidate compounds for use in treating elastin fiber injuries, such as those injuries caused by chronic obstructive pulmonary disease (COPD), chronic bronchitis, emphysema, refractory asthma, and other related diseases.

### BACKGROUND OF THE INVENTION

Lung elastin degradation occurs with the development of pulmonary emphysema in patients with Chronic Obstructive Pulmonary Disease (COPD) related to smoking or alpha-1 antitrypsin deficiency.

Desmosine and Isodesmosine (D and I), the crosslinking amino acids present only in elastin in the human, offer the prospect of assessing elastin degradation in disease by their measurement in certain body fluids. Thus far, D and I have been measured in urine of patients with COPD and found to be statistically significantly elevated above normal controls. One study demonstrated the daily variability of excretion of desmosine and isodesmosine and did not show a statistically significantly elevated excretion of these amino acids in patients in 24-hour collections. In this same study, statistically significantly increased excretion of desmosine and isodesmosine was found in patients with cystic fibrosis.

In addition, peptides of elastin have been measured in plasma by radioimmunoassay (RIA) and found to be elevated in patients with COPD. Because of variability of the specificity of antibodies to elastin peptides in such RIAs, however, quantitation of peptides has varied among various studies. Furthermore, direct measurements of D and I in plasma have not been recorded in normal subjects or patients with COPD and measurements of D and I in sputum have only recently been reported.

In view of the foregoing, there is a need for methods of accurately detecting and measuring elastin components, such as desmosine, isodesmosine or combinations thereof, for the purpose of diagnosing and/or treating COPD, chronic bronchitis, emphysema, refractory asthma, and other related diseases. Similarly, there is a need for methods of validating whether a candidate compound is effective to treat, prevent, or ameliorate the effects of a disease characterized by elastic fiber injury.

### SUMMARY OF THE INVENTION

According to one preferred embodiment of the present invention, methods are provided for validating whether a candidate compound is effective to treat, prevent, or ameliorate the effects of a disease characterized by elastic fiber injury. In such embodiments, the methods comprise determining if the candidate compound decreases the degradation of elastic fiber in a patient administered the candidate compound by measuring, using mass spectrometry, a marker of elastic fiber degradation in a sample of a body fluid or a tissue of the patient. The invention provides that a decrease in the presence of the marker compared to a control validates that the candidate compound is effective to treat, prevent, or ameliorate the disease.

According to another preferred embodiment of the present invention, methods are provided for validating whether a candidate compound is effective to treat, prevent, or ameliorate the effects of COPD. Such methods comprise determining if the candidate compound decreases the degradation of elastin in a patient administered the candidate compound by measuring, using mass spectrometry, the amount of desmosine, isodesmosine, or combinations thereof in a sample of a body fluid or tissue of the patient. The invention provides that a decrease in the presence of desmosine and/or isodesmosine compared to a control validates that the candidate compound is effective to treat, prevent, or ameliorate the disease. In certain preferred embodiments, the body fluid may comprise plasma, urine, sputum, or combinations thereof.

According to additional embodiments of the present invention, methods are provided for identifying candidate compounds that are effective to treat, prevent, or ameliorate the effects of a disease characterized by elastic fiber injury. Such methods of the invention comprise (a) administering a candidate compound to a cell culture model of the disease; (b) measuring, by mass spectrometry, the amount of a marker of elastic fiber injury in the cell culture administered the candidate compound; and (c) determining whether the amount of the marker produced by the cell culture administered the candidate compound is different compared to a control cell culture absent the candidate compound. Non-limiting examples of appropriate markers include desmosine, isodesmosine, or combinations thereof. The invention provides that a decrease in the amount of such marker(s) produced by the cell culture administered the candidate compound compared to the control cell culture identifies the candidate compound as effective to treat, prevent, or ameliorate the effects of the disease.

### BRIEF DESCRIPTION OF THE FIGURES

FIG. 1A: HPLC separation of D and I was achieved by an Atlantis dC18 column (2.1×150 mm, 3 μm) (Waters). The mobile phase A is aqueous 7 mM heptafluorobutyric acid and 5 mM ammonium acetate, and the mobile phase B is a solution of 7 mM heptafluorobutyric acid and 5 mM ammonium acetate in a acetonitrile/water (8:2). HPLC chromatography was performed using a 12 minute linear gradient flow of the mobile phase A from 100% to 88% and mobile phase B from 0% to 12% at a flow rate of 0.3 ml/min. The temperature of the HPLC column is set at 30° C. Under these chromatographic conditions desmosine and isodesmosine were detected at 8.95 minutes and 9.90 minutes, respectively. The mass spectrometer was operated in the positive ion mode with the following spectrometric parameters: capillary voltage 3.20 kV, sample cone voltage 55 V, ion energy 0.5 eV, amplifier voltage 650 V, and temperatures of the desolvation and the source at 400° C. and 120° C., respectively.

FIG. 1B: Quantification of D and I was achieved by a single ion record (SIR) of D and I molecular ions, both at m/z 526.25 (two isomeric molecules), produced from the LC/MS analysis. Peak areas of the SIR obtained by D and I standards provided good linearity between 0.05 ng to 5 ng.

FIG. 2: Shown in FIG. 2 are mean and standard error of the mean of D and I in plasma for normal controls, patients with COPD without alpha-1 antitrypsin deficiency (AATD) and patients with COPD with AATD. The differences among all three groups are statistically significant. P-values are calculated based on the summed values of D and I using the unpaired t-test with Welch's correction.

FIG. 3: Shown in FIG. 3 are mean levels and standard errors of the mean of D and I in urine of normal controls, patients with COPD without AATD and COPD with AATD. Mean differences among the groups are statistically significant for the free D and I and % of free/total D and I urinary excretion. P-values calculated as in FIG. 1.

FIG. 4: Shown in FIG. 4 are mean levels and standard error of the mean of D and I in sputum of patients with COPD without AATD and COPD with AATD. Control subjects do not have detectable D or I in induced sputum. The content of D and I in sputum of patients with COPD and AATD is statistically significantly higher than in those with COPD and without AATD. P-values calculated as in FIG. 1.

FIG. 5: A table summarizing the experimental results described herein relative to COPD patients having normal alpha 1-antitrypsin.

FIG. 6: A table summarizing the experimental results described herein relative to patients having alpha 1-antitrypsin related-COPD.

FIG. 7: FIG. 7 demonstrates increasing specificities of three analytical methods. The HPLC/UV method measures all molecular species that have the same UV absorption (286 mμ) with D and I. The SIM method identifies and quantifies all molecules that have the same molecular weight (526) with D and I. The RIM method identifies and quantifies the ion fragments (481 and 397) that are only derived from D and I.

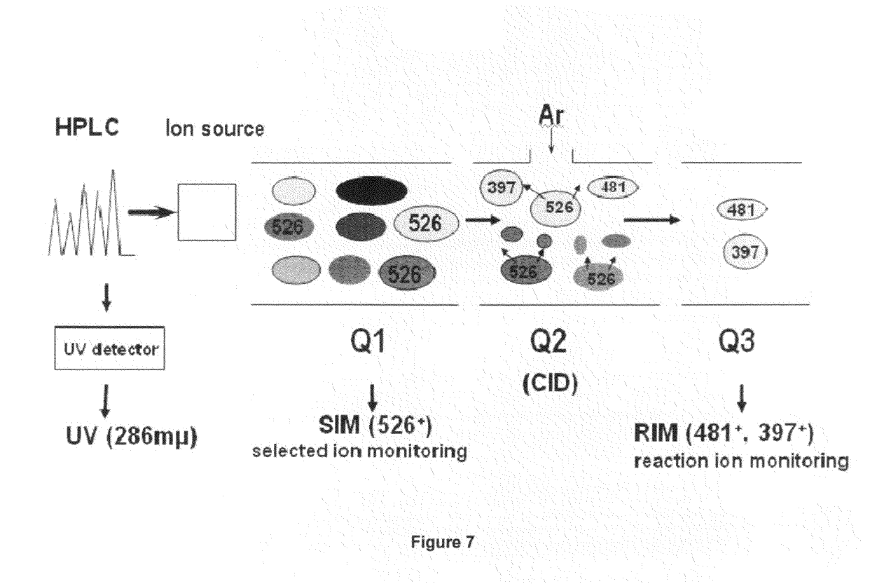

FIG. 8: Shown in FIG. 8 are measurement of D and I in plasma using various methods (HPLC/UV (A), SIM (B), and RIM (C)) described in FIG. 7. This figure demonstrates increasing specificity or sensitivity of the methods using a sample of 0.3 ng D/I in 0.5 mL of COPD plasma. The mass spectrometric method (LC/MS and LC/MS/MS) for the measurement of D/I in urine, plasma and sputum is more sensitive and specific than existing radioimmunoassays and HPLC methods.

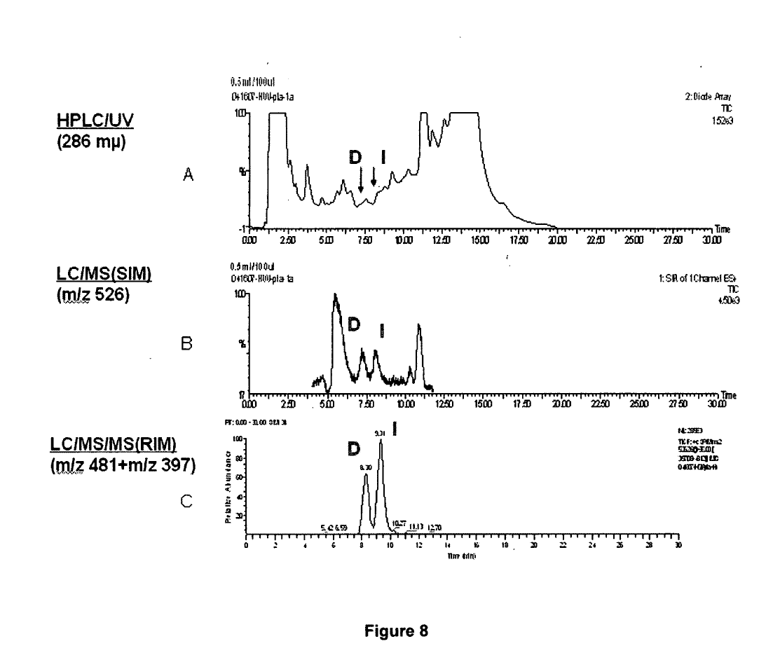

FIG. 9: A table summarizing the experimental results described herein relative to patients treated with Tiotropium.

FIG. 10: Shown in FIG. 10 are percent decrease in D/I levels in urine, plasma, and sputum from patients before treatment and two months after treatment with Tiotropium.

### DETAILED DESCRIPTION OF THE INVENTION

According to one preferred embodiment of the present invention, methods are provided for validating whether a candidate compound is effective to treat, prevent, or ameliorate the effects of a disease characterized by elastic fiber injury, such as elastin degradation. In such embodiments, the methods comprise determining if the candidate compound decreases the degradation of elastic fiber in a patient administered the candidate compound by measuring, using mass spectrometry, a marker of elastic fiber degradation in a sample of a body fluid or a tissue of the patient. The invention provides that a decrease in the presence of the marker compared to a control validates that the candidate compound is effective to treat, prevent, or ameliorate the disease.

The foregoing methods may be used to validate whether a candidate compound is effective to treat, prevent, or ameliorate the effects of chronic obstructive pulmonary disease (COPD), chronic bronchitis, emphysema, and/or refractory asthma. The marker of elastic fiber degradation that is measured using mass spectrometry is preferably desmosine, isodesmosine, or combinations thereof. In such embodiments, the marker(s), such as desmosine, isodesmosine, or combinations thereof, are preferably detected and measured within a patient's urine, plasma, and/or sputum.

In certain preferred embodiments of the invention, desmosine, isodesmosine, or combinations thereof are measured in plasma. In certain alternative embodiments, total free desmosine, isodesmosine, or combinations thereof are measured in urine. The methods of the present invention may be employed to test the therapeutic value, or effectiveness, of a variety of different candidate compounds. Non-limiting examples of such compounds include hyaluronic acid, polysaccharides, carbohydrates, small molecules, and RNAi molecules, including siRNAs, shRNAs, and others.

According to additional embodiments of the present invention, methods are provided for identifying candidate compounds that are effective to treat, prevent, or ameliorate the effects of a disease characterized by elastic fiber injury. Such methods of the invention comprise (a) administering a candidate compound to an in vivo or in vitro model of the disease, e.g., a cell culture; (b) measuring, by mass spectrometry, the amount of a marker of elastic fiber injury in the cell culture administered the candidate compound; and (c) determining whether the amount of the marker produced by e.g., the cell culture administered the candidate compound is different compared to e.g., a control cell culture absent the candidate compound. Non-limiting examples of appropriate markers include desmosine, isodesmosine, or combinations thereof. The invention provides that a decrease in the amount of such marker(s) produced by e.g., the cell culture administered the candidate compound compared to e.g., the control cell culture identifies the candidate compound as effective to treat, prevent, or ameliorate the effects of the disease.

Such methods may be used for identifying candidate compounds that are effective to treat, prevent, modulate and/or ameliorate the effects of elastin degradation and diseases associated therewith, such as COPD, chronic bronchitis, emphysema, and/or refractory asthma. Similar to the other embodiments discussed herein, the marker that is measured by mass spectrometry is preferably selected from desmosine, isodesmosine, or combinations thereof. Still further, similar to the other embodiments discussed herein, such methods may be employed to test the therapeutic value, or effectiveness, of a variety of different candidate compounds, including hyaluronic acid, polysaccharides, carbohydrates, small molecules, and RNAi molecules, such as siRNAs, shRNAs, and others.

The following examples are provided to further illustrate the methods of the present invention. These examples are illustrative only and are not intended to limit the scope of the invention in any way.

### EXAMPLES

In these examples, measurements of desmosine (D) and isodesmosine (I) in plasma, urine and sputum are described. The results demonstrate a statistically significant difference between normal controls and patients diagnosed with COPD and further suggest that measurements of D and I in plasma may be a discriminating index distinguishing patients with COPD from normal subjects. D and I were measured in plasma, urine and sputum in a cohort of patients diagnosed with COPD related to smoking and a second cohort in whom COPD is related to Z-phenotype alpha-1 antitrypsin deficiency (AATD) as well as smoking.

### Example 1

### Materials and Methods

The mass spectrometric method was used for direct measurement of D/I in urine, plasma and sputum as markers of elastin degradation in healthy controls, patients with α1-antitrypsin deficiency (AATD) and non-AATD-related COPD. Preparation of specimens of urine and sputum and measurements by mass spectrometry (LC/MS) were performed as previously described in Ma S, Lieberman S, Turino G M and Lin Y Y: The detection and quantitation of free desmosine and isodesmosine in human urine and their peptide-bound forms in sputum. PNAS 2003, 100:12941-12943, which is incorporated by reference as if recited in full herein. D and I standard (mixed 50% D and 50% I) were purchased from Elastin Products (Owensville, Mich.), and all other reagents were from Sigma (St. Louis, Mo.). MCX cation exchange cartridges (3 ml) were obtained from Waters (Milford, Mass.), and CF1 cellulose powders were purchased from Whatman (Clifton, N.J.).

Urine samples. Twenty-four hour urine samples were collected and analyzed as previously described in Ma S, et al., 2003.

Plasma samples. Plasma samples were obtained after centrifuging venous blood specimens at 2500 r.p.m. for 25 min. Samples were stored at −20 C until used. One ml of plasma and 1 ml of concentrated HCl (37%) were placed in a glass vial. After air in the sample was displaced with a stream of nitrogen, the sample was acid hydrolyzed for 24 hours in 6N HCl. After evaporation to dryness, the residue was dissolved in 2 ml of a mixed solution of n-butanol/acetic acid/6 N HCl (4:1:1, by volume). The sample solution was loaded onto a 3 ml CF1 cartridge. The CF1 cartridge was prepared by introducing 3 ml of the slurry of 5% CF1 cellulose powder in a mixture of n-butanol/acetic acid/water (4:1:1, by volume). The cartridge was washed 3 times with 3 ml of n-butanol/acetic acid/water mixture, and D and I adsorbed in the CF1 cartridge were eluted with 3 ml of water. The eluate was evaporated to dryness under vacuum at 45° C. and the residue was dissolved in 0.1 ml of HPLC mobile phase for LC/MS analysis. For analysis in plasma, samples were processed and measured in duplicate and the results averaged.

Sputum Samples. Sputum samples were processed as previously described in Ma S, et al., 2003 with the following modification: The acid hydrolyzed samples were chromatographed using a CF1 cartridge as described in the treatment of plasma samples. Each sputum sample was processed and measured in duplicate and the results averaged. Sputum was obtained from 3-hour morning collections spontaneously produced. When subjects could not voluntarily produce sputum, sputum induction was induced by 3% saline inhalation for 20 minutes as previously described in Ma S, et al., 2003.

Recovery of Desmosine and Isodesmosine in Urine and Plasma. Using D and I as the external standards we performed studies to ensure recovery and reproducibility of the analysis in urine and plasma. Triplicates of two urine samples, were spiked with 0.4 pmol and 2.0 pmol each of D and I standards, and carried through HCl hydrolysis and chromatography procedures as described. The recoveries of D and I from one urine spiked with 2.0 pmol of D and I were 91±4% and 88±1%, and that spiked with 0.4 pmol of D and I were 92±3% and 93±8%. The recoveries of D and I from the other urine spiked with 2.0 pmol of D and I were 88±1% and 93±3%, and that spiked with 0.4 pmol of D and I were 93±6% and 93±15%. The reproducibility of the repeated sample analysis ranges from 91-99%.

Similar recovery studies were carried out with 4 plasma samples. The recoveries of D and I with 0.05 ng standards were 65±4 and 74±13%, and that with 0.1 ng standards were 67±1 and 72±4%. The reproducibility of the repeated sample analysis is 83-99%. Values in urine and plasma were corrected for recovery losses.

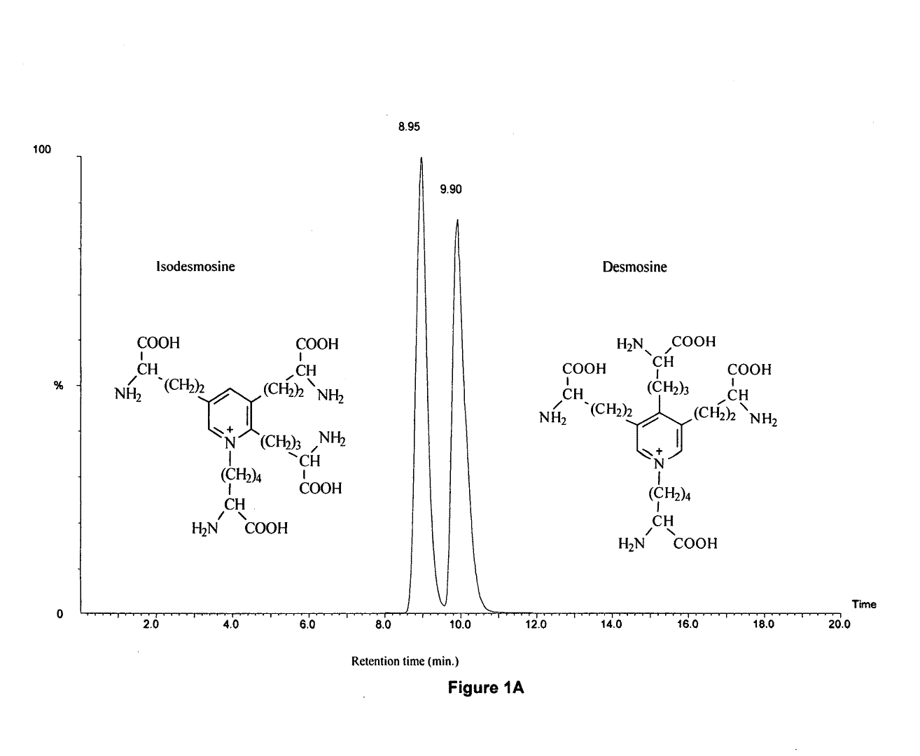

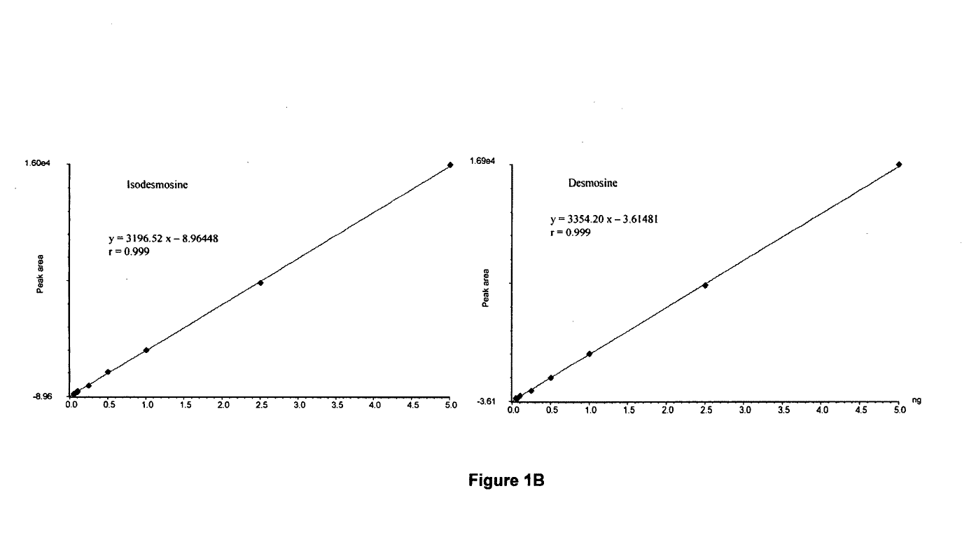

Creatinine and Protein Measurement were carried out as previously described in Ma S, et al., 2003. LC/MS Analysis was performed also as previously described in Ma S, et al., 2003, with slight modification (see Legend to FIG. 1A).

Data Analysis. The t-test adjusted for unequal variance was used to test the null hypothesis. The level of significance was 0.05. P-values were calculated based on the summed values of D and I using the unpaired t-test with Welch's correction.

Patients. Study patients were diagnosed with chronic obstructive pulmonary disease and adhere to Gold Criteria grades 1-4. All patients were screened for alpha-1 antitrypsin deficiency (AATD) by serum levels and phenotyping. Patients were divided into two groups: 1) with normal levels of alpha-1 antitrypsin in serum and 2) those with ZZ-homozygous alpha-1 antitrypsin deficiency. Patients gave informed consent for the study. The study was approved by the Institutional IRB.

All patients with normal levels of alpha-1 antitrypsin had significant smoking histories of from 10 to 60 pack years. Many had stopped in the previous ten years and none were current smokers when studied. Among these patients the age range was 44 to 85. Five were males and 2 females.

Among patients with alpha-1 antitrypsin deficiency all but one had a significant smoking history exceeding ten pack years. All patients had ceased smoking for at least ten years by the time of study. All AATD patients were being treated with AAT protein replacement, were in a stable clinical state and exhibited no evidence of an exacerbation.

Control subjects were selected by a clinical history free of any specific known disease or significant symptoms, including respiratory symptoms, and none had ever smoked.

### Example 2

### Results

Results in normal subjects are presented in Table 1 below (C=Caucasian; A=Asian).

TABLE 1
Controls without Lung Disease

| | | | | Desmosine/Isodesmosine | | | | |
|---|---|---|---|---|---|---|---|---|
| | | | | | | Urine | | |
| | | | | Plasma | | Free Form | | |
| | | | | | ng/g | μg/g | | Free/Total |
| Subjects | Sex | Age | Race | ng/ml | protein | creatinine | Total | % |
| 1 | M | 33 | C | 0.11/0.10 | 1.89/1.80 | 1.85/1.11 | 9.64/5.90 | 19/19 |
| 2 | M | 35 | C | 0.07/0.09 | 1.06/1.36 | | | |
| 3 | F | 58 | C | 0.10/0.08 | 2.17/1.74 | 3.73/2.76 | 10.22/7.65 | 36/36 |
| 4 | M | 27 | A | 0.09/0.07 | 1.31/1.02 | 0.60/0.50 | 2.85/2.70 | 21/19 |
| 5 | F | 31 | A | 0.10/0.06 | 2.22/1.29 | | | |
| 6 | F | 69 | C | 0.09/0.08 | 1.62/1.44 | 1.80/1.69 | 11.77/8.37 | 15/20 |
| 7 | M | 54 | A | 0.11/0.13 | 2.02/2.27 | 0.51/0.64 | 5.17/3.96 | 10/16 |
| 8 | M | 72 | A | 0.09/0.13 | 1.94/2.80 | 0.75/0.35 | 5.16/4.10 | 15/9 |
| 9 | M | 79 | C | 0.12/0.05 | 2.43/1.01 | 0.42/0.38 | 6.17/4.67 | 7/8 |
| 10 | M | 65 | A | 0.11/0.10 | 2.23/2.03 | 0.99/0.66 | 6.59/3.89 | 15/17 |
| 11 | F | 38 | A | 0.13/0.08 | 2.27/1.31 | 0.89/0.88 | 5.19/4.26 | 17/21 |
| 12 | F | 28 | C | 0.11/0.09 | 1.83/1.50 | 2.48/1.58 | 12.69/6.64 | 20/24 |
| 13 | M | 32 | C | 0.10/0.08 | 1.87/1.49 | 1.59/1.56 | 7.29/5.68 | 22/27 |
| mean | | | | 0.10/0.09 | 1.91/1.62 | 1.42/1.10 | 7.52/5.26 | 18/20 |
| ±SEM | | | | ±0.01/±0.01 | ±0.11/±0.14 | ±0.31/±0.22 | ±0.94/±0.53 | ±2/±2 |

The mean levels and standard error (S.E.M.) of D and I (D/I) in plasma in 13 subjects were 0.10±0.01/0.09±0.01 ng/ml plasma and 1.91±0.11/1.62±0.14 ng/g protein.

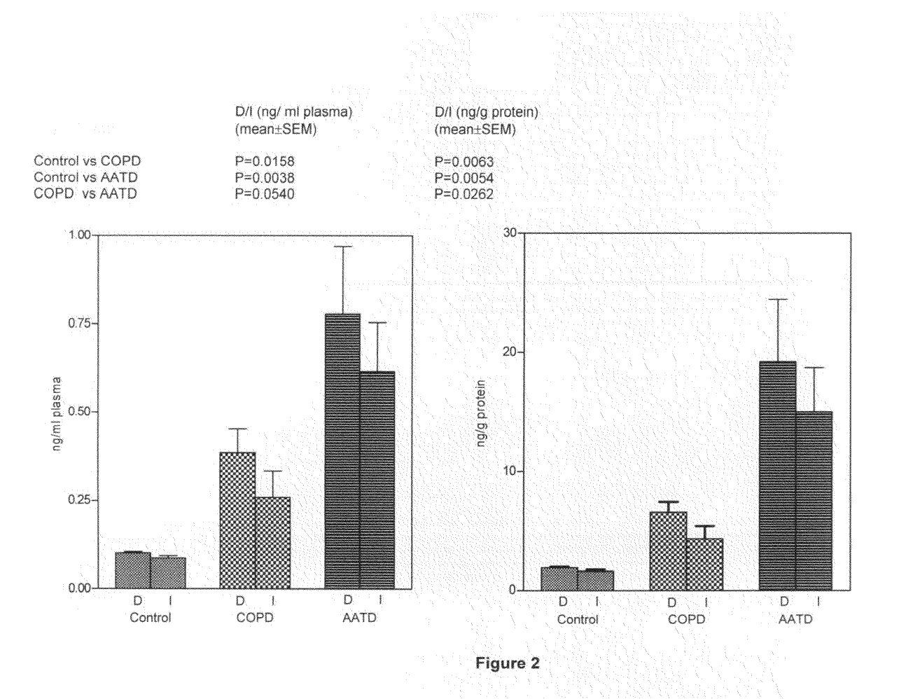

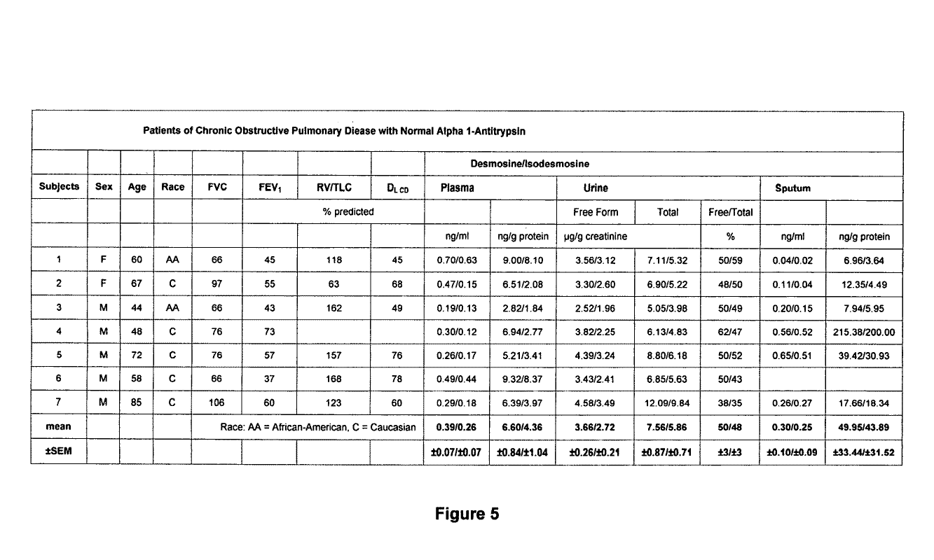

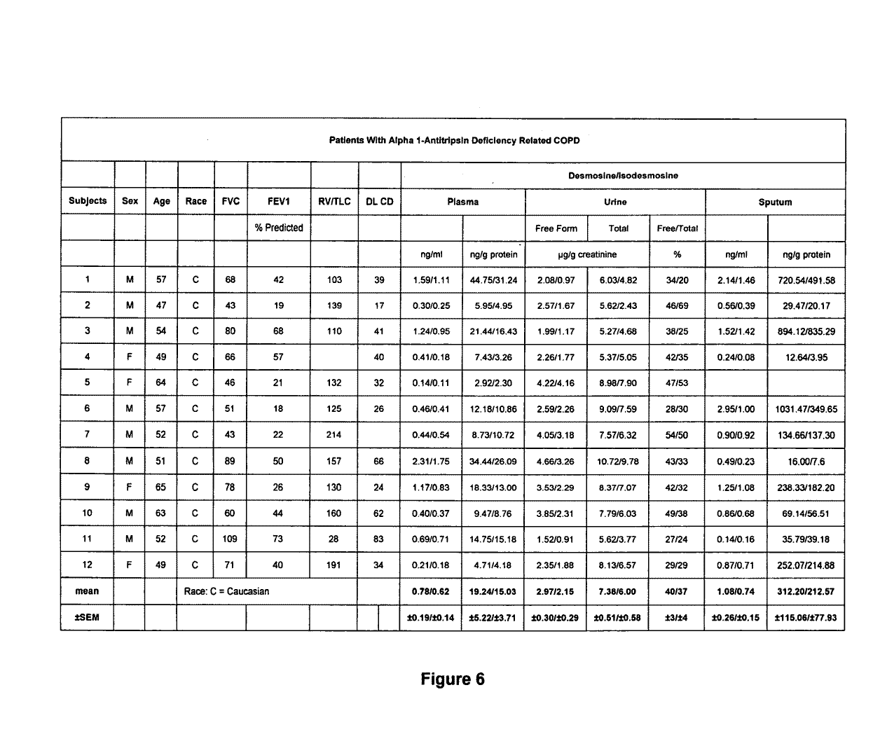

Results for levels of D and I (D/I) in plasma in patients with COPD with normal levels of AAT are presented in FIGS. 2 and 5. The mean and S.E.M. were 0.39±0.07/0.26±0.07 ng/ml of plasma and 6.60±0.84/4.36±1.04 ng/g protein, which are statistically significantly higher than controls. Results for levels of D and I in plasma in patients with COPD related to AATD are shown in FIGS. 2 and 6. The mean and S.E.M. are 0.78±0.19/0.62±0.14 ng/ml of plasma and 19.24±5.22/15.03±3.71 ng/g protein, which values are statistically significantly higher than control values and the levels in COPD not related to AATD when calculated per gm of protein in plasma.

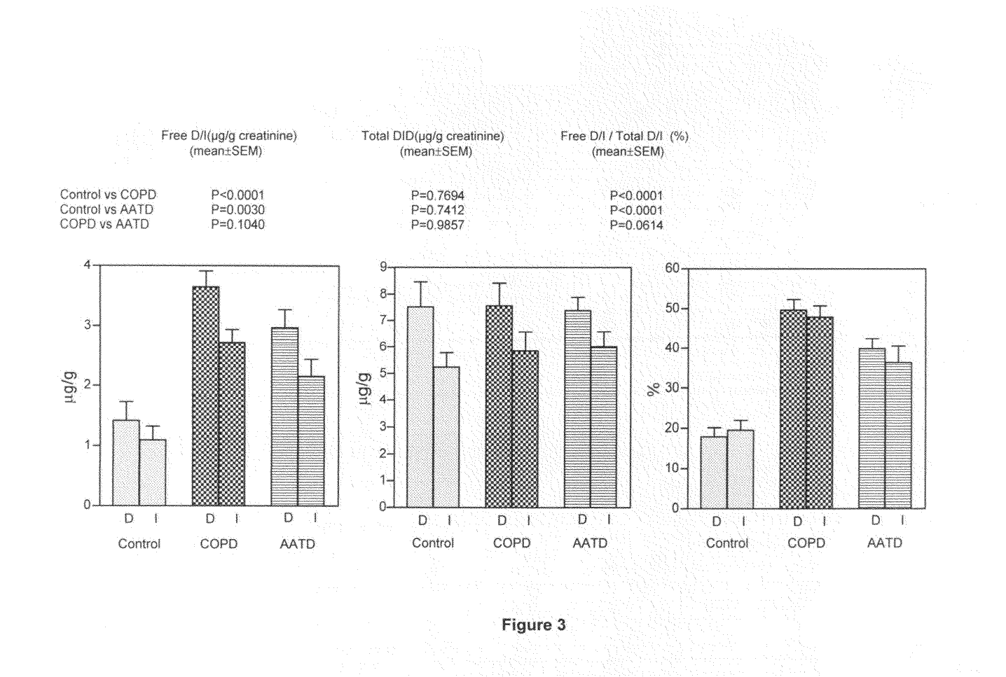

It is noteworthy that no overlap of levels of plasma D and I exists between controls and the patient groups with COPD; patients' levels are consistently higher. The levels of D and I in urine in control subjects and patients with and without AATD are shown in Table 1 and FIGS. 3, 5 and 6. The levels of free D and I (D/I) are 3.66±0.26/2.72±0.21 ng/g creatinine in COPD with normal levels of AAT and 2.97±0.30/2.15±0.29 in patients with AATD which values are statistically significantly higher than control subjects (1.42±0.31/1.10±0.22). As shown in FIG. 3, the percentage of free D and I over total D and I excretion was statistically significantly higher in both groups with COPD, but highest in COPD with normal AAT levels. The mean total 24 hour excretion of D and I was not statistically significantly increased in both COPD groups as compared to controls.

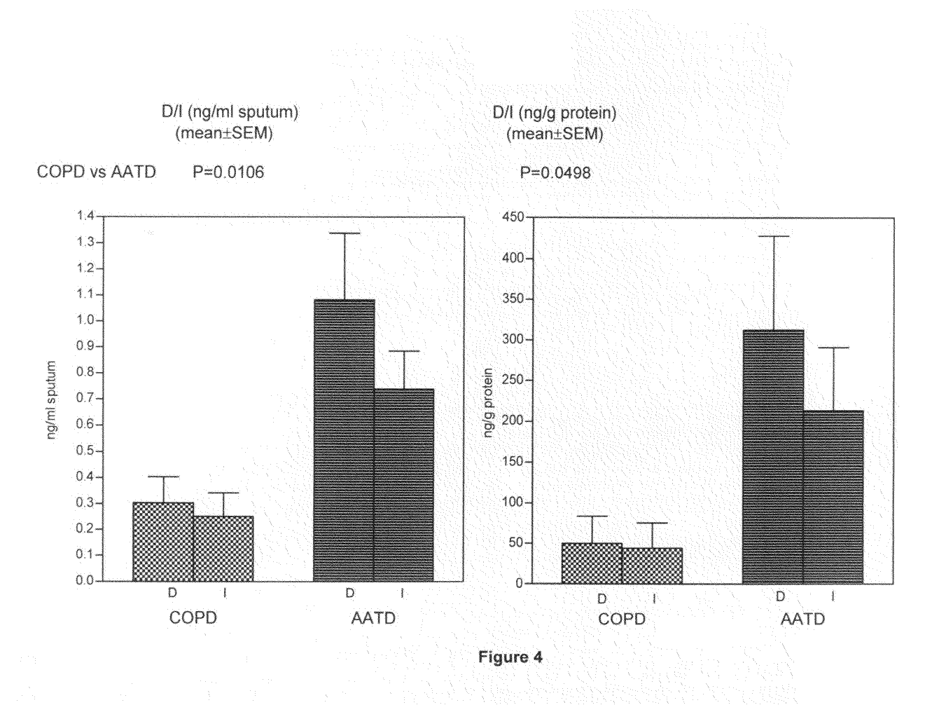

Levels of D and I in sputum are shown in FIGS. 4-6. The levels of D and I were below the level of detection by mass spectrometry in 3 control subjects, whereas both groups of COPD patients showed mean levels of D and I to be significantly increased to 1.08±0.26/0.74±0.15 ng/ml and 0.30±0.10/0.25±0.09 ng/ml of sputum in COPD patients with and without AATD respectively. Results expressed per g of protein in sputum for D and I (D/I) were 312±115/212±77.9 and 49.9±33.4/43.9±31.5 in patients with and without AATD. D and I in sputum was statistically significantly higher in AATD patients.

Shown in Table 2 below are repeat measurements of plasma D and I in 1 control subject, 1 patient with AATD related COPD and a patient with COPD without AATD. Intervals between repeat measurements were days in subjects with AATD and COPD to weeks and months for the other two subjects. During these intervals, each patient was in a stable clinical state without exacerbations.

TABLE 2
Repeat Measurements of Desmosine and Isodesmosine in Plasma

| | |
|---|---|
| D/I (ng/ml) | D/I (ng/g protein) |

Normal Subject - 14 month interval

| | |
|---|---|
| 0.12/0.05 | 2.43/1.01 |
| 0.11/0.07 | 2.11/1.34 |

Patient with COPD and AATD - 2 day interval

| | |
|---|---|
| 2.31/1.75 | 54.91/41.60 |
| 2.53/2.08 | 55.90/45.96 |
| 2.55/2.49 | 61.31/59.87 ng/g protein |

Patient with COPD without AATD - 6 month interval

| | |
|---|---|
| 0.49/0.44 | 9.32/8.37 |
| 0.32/0.31 | 7.04/6.82 |

The results varied between 10 and 15%, which suggests a stable metabolic state with respect to elastin turnover in each individual's normal or abnormal levels.

Levels of D and I (D/I) in plasma and urine were analyzed for possible correlation with age, sex, racial origin or physiological parameters of FEV₁ and RV/TLC and no statistically significant correlations were determined.

### Example 3

### Data Analysis

An early insight into the mechanisms leading to alveolar disruption in pulmonary emphysema is that lung matrix elastin is a target for chemical degradation from cellular elastases. Lung elastin content, determined chemically, has been demonstrated to be low in pulmonary emphysema related to smoking or to the Z-phenotype AATD, and morphologically, lung elastin fibers have been shown to be fragmented and disordered. Also intratracheal administration of elastases has uniquely produced animal models of pulmonary emphysema. In addition, elastin peptides have been shown to be chemotactic for neutrophils and macrophages and could be a factor in the progression of human pulmonary emphysema once elastin degradation has occurred.

Current methods of measuring elastin peptides in blood plasma require radioimmunoassay techniques which depend on antibodies to elastin peptides which vary in specificity and sensitivity, which affects the standardization and quantification of peptides. Also, measurements of D and I in urine require a relatively extensive chemical procedure using isotope dilution corrections and HPLC, which can be an arduous methodology.

Recognizing these limitations, mass spectrometry, with its ability to detect specific molecular species with high sensitivity, accuracy and specificity is a readily applicable method for use in complex body fluids. The increased sensitivity of mass spectrometry has permitted the measurement of a free component unbound to protein or other matrix constituents of D and I in urine which are increased statistically significantly in patients with COPD as compared with normals. Similarly, mass spectrometry has allowed measurements of D and I in blood plasma and sputum, both chemically complex media. Attempts to detect a free vs bound component of D and I in plasma were unsuccessful. The concentration of D and I in a single small sample of plasma may be too low for detection compared to the concentration of D and I in a 24-hour collection of urine.

The increased free component of D and I in urine in COPD patients, we believe, may reflect an increased neutrophil elastase concentration in circulating neutrophils, which has been demonstrated by previous measurements as an increase in lysosomal elastase in neutrophils of COPD patients as compared with normals. This increased elastase concentration may reflect a generalized immunological hyperreactivity resulting from the chronic inflammatory state of the lung in COPD, manifested by increased elastase activity in neutrophils and macrophages.

The difference in levels of D and I in plasma between controls and patients with COPD in this study suggests that D and I in plasma may be one of the sensitive indicators of the presence of lung elastin breakdown in COPD, especially since the entire cardiac output constantly circulates through the lung. While changes in levels of D and I in plasma cannot be assumed to reflect D and I from lung parenchyma per se, the demonstrated presence of D and I in sputum of patients with COPD indicates that increased degradation, and probably turnover, of elastin is occurring in lung, since normal subjects do not have detectable amounts of D and I in induced sputum.

In the limited number of our controls we did not find any correlation of the age of the subjects with urinary excretion or plasma levels of D and I. In other studies of adult subjects which include similar measurements no correlations with age have been reported.

Measurements of total excretion of D and I in 24 hour urine collection did not demonstrate statistically significant differences between patients and normals. This result is consistent with the demonstration of Bode et al., who showed marked variability in daily excretion of D and I in COPD patients and no statistically significant difference in the total excretion between the two cohorts. (Bode D C, Pagan E D, Cuminskey R, von Roemaling R, Hamel L and Silver P J: Comparison of urinary desmosine excretion in patients with chronic obstructive disease or cystic fibrosis. Pul Pharmacol Ther 2000, 13:175-180). Also, Starcher et al. have demonstrated a failure of urine to reflect the rapid degradation of lung elastin produced by intratracheal porcine pancreatic elastase in mice. Their studies demonstrated a sequestering of elastin peptides in renal parenchyma following lung elastin breakdown and a continued slow urinary excretion of D containing peptides over several days following acute elastase injury. (Starcher B and Peterson B: The kinetics of elastolysis: elastin catabolism during experimentally induced fibrosis. Exp Lung Res 1999, 25:407-424). Other studies have shown significant increases of urinary D in COPD patients compared to normals. Possibly the individual patient population in the present study varied from those previously studied. In that regard, none of the patients in this study were actively smoking, which has been shown to increase urinary desmosine excretion.

When elastin degradation is mildly, or even moderately, increased above the turnover in normals, it may be difficult to reflect this increase in urine, even with 24-hour collections. However, the percentage of the free component of D and I in urine is consistently elevated in both groups of patients with COPD.

It has long been demonstrated that elastin in elastin fibers, once formed, cross-linked and insoluble, is extremely stable and undergoes little metabolic turnover. This slow metabolic turnover in normal humans is consistent with the very low levels of D and I in normal plasma. It is noteworthy that studies of elastase injury to lung elastin in vivo in rats and mice demonstrate that rapid degradation of elastin occurs when exposed to elastases, with rapidly ascending concentrations of elastin peptides in blood and urine within hours of protease administration. Notable also is the rapid resynthesis of elastin after proteolytic breakdown. The stability of plasma and urine levels of desmosine with repeat measurements over a 44 day interval in patients with AATD was reported by Stolk et al., which is consistent with measurements in this study. (Stolk J, Veldhuisen B, Annovazzi L, Zanone C, Versteeg E M, van Kuppevelt, T H, Nieuwenhuizen, W, Iadarola P and Luisetti M: Short-term variability of biomarkers of proteinase activity in patients with emphysema associated with type Z alpha-1 antitrypsin deficiency. Respir Res 2005, 6:47). Thus any increase in elastase activity in lungs, which includes bronchial and blood vessel elastin as well as alveolar, may well be reflected in the circulating blood to and from the lung.

The persistence of elevated levels of D and I in plasma in patients with COPD in both patient cohorts long after smoking cessation is consistent with continued inflammation of the lung in COPD and progression of matrix tissue injury.

The blood levels of D and I in COPD patients may therefore prove to be a sensitive index of the metabolic state of elastin degradation and possibly resynthesis in the lung. Since elastin is a significant structural constituent of alveoli, bronchial walls and blood vessels, the levels of D and I in the earliest phases of COPD deserve to be evaluated. Also the responses to therapeutic agents which may reduce the lung inflammatory state and thereby reduce elastin degradation may be assessed by measurements of D and I in plasma and the proportion of free D and I in urine.

It is noteworthy that the ATTD patients had higher levels of D and I in plasma than COPD patients without AATD, along with higher levels in sputum consistent with the ATTD patients' form of COPD to be emphysematous with loss of lung mass. All patients with AATD were receiving AAT augmentation therapy at the time of study. Since levels of D and I in body fluids were not obtained prior to the initiation of augmentation therapy, it cannot be assumed that AAT replacement is having no beneficial effect. These data suggest that an evaluation of the effect on D and I levels of higher doses of AAT augmentation would be worthwhile.

Mass spectrometry allows measurements of D and I separately. The proportion of D and I in plasma and urine in control subjects shows a slightly lower proportion of isodesmosine constituting approximately 80% of the level of desmosine. In one study of the amino acid composition of human lung elastin, D exceeded I content by approximately 10-15%, which is close to agreement with the present study. (Starcher B C and Galione M J: Purification and comparisons of elastin from different animal species. Analytical Biochemistry 1976, 74:441-447). It is noteworthy that patients with COPD in both groups had proportions of D and I which are similar to controls, suggesting that resynthesis of elastin in these groups does not show major structural dissimilarities from normals.

The results of this study indicate that levels of D and I in urine which includes an unconjugated fraction, along with levels in plasma and sputum may be useful parameters to characterize patients with COPD of various phenotypes at various phases of the disease. Mass spectrometry, with its increased specificity and sensitivity, should facilitate this characterization.

### Example 4

### Effect of Tiotropium Treatment

COPD patients have elevated levels of D/I in plasma, urine and sputum, which might respond to prolonged bronchodilation. To determine if clinical effects of Tiotropium (TIO) affect tissue degradation of the lung in COPD, clinically stable patients with COPD (n=9) not on TIO prior to the study and at one month and a second month after initiating therapy were tested. Other anticholinergic bronchodilators were stopped prior to TIO, and other therapies/disease treatments were unchanged for the two months of study. To these patients, 18 mcg TIO was administered each 24 hours. D/I in plasma, urine and sputum were measured by liquid chromatography and mass spectrometry (LC/MS) prior to the study and at one month and two months after the study.

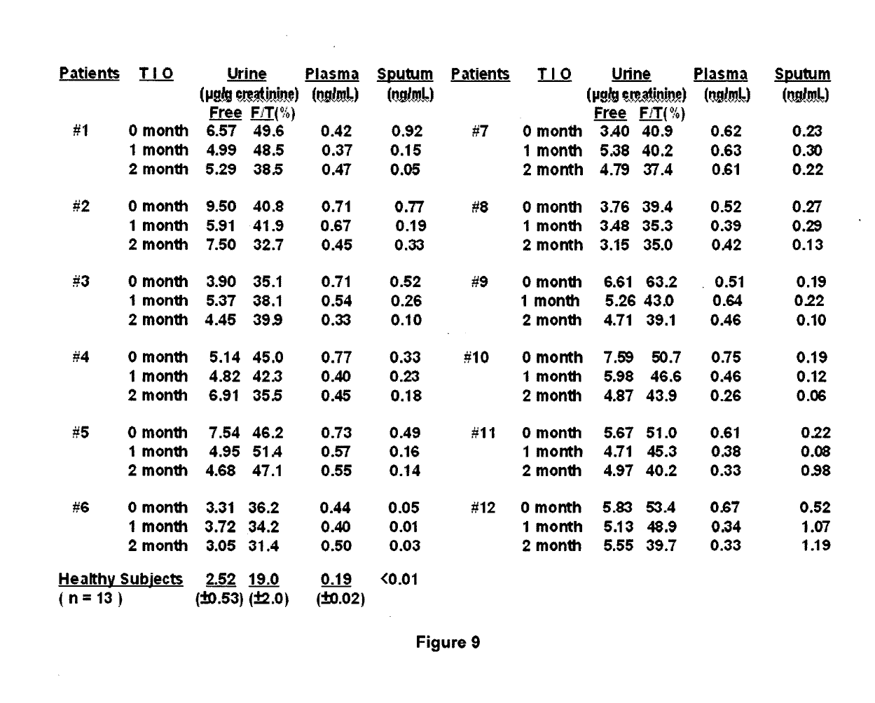

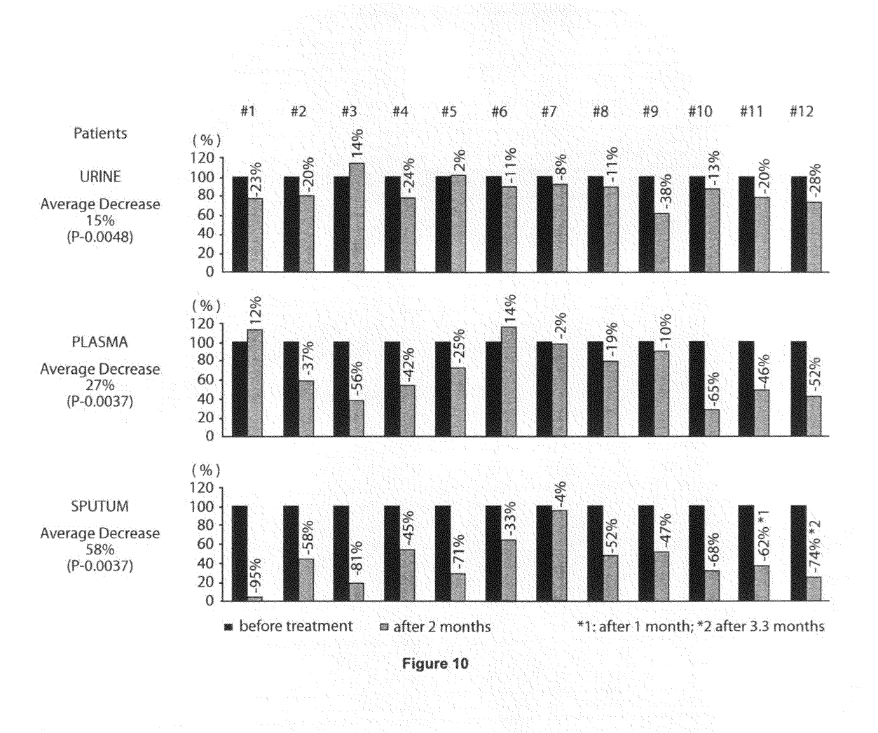

Prior to the study, levels of D/I in plasma and sputum were above normal in all patients studied, and the percentage of free D/I in urine was also increased. Significant decreases in D/I levels were observed in urine (10 out of 12), in plasma (10 out of 12) and in sputum (all 12 patients), which may reflect decreases in lung elastin degradation of COPD patients on TIO therapy. (FIG. 9). Calculated percentage decreases in D/I levels after TIO treatment showed decreases beginning after one month with further decreases observed in the second month. After two months of treatment, larger decreases in D/I levels were observed in sputum and plasma than urine. The response was not always uniform in the respective patients' urine, plasma, and sputum. For example, two patients (#3 and #5) failed to show responses in urine but showed decreases in their plasma and sputum, and two other patients (#1 and #6) did not show decreases in plasma but showed decreases in urine and sputum. (FIG. 10).

Overall results of percent decreases in D/I levels indicated that all 12 COPD patients were responding to prolonged TIO treatment with some decrease in lung elastin degradation. Spirometry in most post-TIO therapy patients shows significant increase in Force Vital Capacity (FVC), Forced Expired Volume in 1 second (FEV1), and ratio of FEV1/FVC and decreases in Residual Volume (RV). The improvement in spirometric indices were usually concordant with levels of D/I in patients.

Overall results demonstrate that two months of treatment with TIO in patients is accompanied by significant reductions in D/I levies in plasma, urine and sputum, consistent with a reduction in elastin degradation and possibly an anti-inflammatory effect. Thus, this example confirms the effectiveness of the methods disclosed and claimed herein for, e.g., validating whether a candidate compound is effective to treat, prevent or ameliorate the effects of a disease characterized by elastic fiber injury, such as COPD, COPD with AATD, chronic bronchitis, emphysema, or refactory asthma.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of validating whether a candidate compound is effective to treat, prevent, or ameliorate the effects of a disease characterized by elastic fiber injury comprising determining if the candidate compound decreases the degradation of an elastic fiber in a patient administered the candidate compound by measuring, using mass spectrometry, a marker of elastic fiber degradation in a sample of a body fluid or a tissue of the patient, wherein a decrease in the presence of the marker compared to a control validates that the candidate compound is effective to treat, prevent, or ameliorate the disease.

2. The method according to claim 1, wherein the elastic fiber injury is elastin degradation.

3. The method according to claim 1, wherein the disease is selected from the group consisting of chronic obstructive pulmonary disease (COPD), COPD with alpha-1 antitrypsin deficiency (AATD), chronic bronchitis, emphysema, and refractory asthma.

4. The method according to claim 1, wherein the disease is COPD.

5. The method according to claim 1, wherein the marker of elastic fiber degradation is selected from the group consisting of desmosine, isodesmosine, and combinations thereof.

6. The method according to claim 1, wherein the marker is both desmosine and isodesmosine.

7. The method according to claim 1, wherein the body fluid is selected from the group consisting of urine, plasma, and sputum.

8. The method according to claim 5, wherein both desmosine and isodesmosine are measured in plasma.

9. The method according to claim 5, wherein total free desmosine and isodesmosine are measured in urine.

10. The method according to claim 1, wherein the candidate compound is selected from the group consisting of hyaluronic acid, polysaccharide, carbohydrate, small molecules, and RNAi.

11. A method of validating whether a candidate compound is effective to treat, prevent, or ameliorate the effects of chronic obstructive pulmonary disease (COPD) comprising determining if the candidate compound decreases the degradation of elastin in a patient administered the candidate compound by measuring, using mass spectrometry, the amount of desmosine and isodesmosine in a sample of a body fluid or tissue of the patient, wherein a decrease in the presence of desmosine or isodesmosine compared to a control validates that the candidate compound is effective to treat, prevent, or ameliorate the disease.

12. The method according to claim 11, wherein the body fluid is selected from the group consisting of urine, plasma, and sputum.

13. The method according to claim 12, wherein both desmosine and isodesmosine are measured in plasma.

14. The method according to claim 12, wherein total free desmosine and isodesmosine are measured in urine.

15. A method of validating whether a candidate compound is effective to treat, prevent, or ameliorate the effects of chronic obstructive pulmonary disease (COPD) comprising determining if the candidate compound decreases the degradation of elastin in a patient administered the candidate compound by measuring, using mass spectrometry, the amount of desmosine and isodesmosine in a sample from the patient selected from the group consisting of plasma, urine, and sputum, wherein a decrease in the presence of desmosine and isodesmosine compared to a control validates that the candidate compound is effective to treat, prevent, or ameliorate the disease.

16. A method for identifying candidate compounds that are effective to treat, prevent, or ameliorate the effects of a disease characterized by elastic fiber injury comprising:
(a) administering a candidate compound to a cell culture model of the disease;
(b) measuring, by mass spectrometry, the amount of a marker of elastic fiber injury in the cell culture administered the candidate compound; and
(c) determining whether the amount of the marker produced by the cell culture administered the candidate compound is different compared to a control cell culture absent the candidate compound, wherein a decrease in the amount of the marker produced by the cell culture administered the candidate compound compared to the control cell culture identifies the candidate compound as effective to treat, prevent, or ameliorate the effects of the disease.

17. The method according to claim 16, wherein the elastic fiber injury is elastin degradation.

18. The method according to claim 16, wherein the disease is selected from the group consisting of chronic obstructive pulmonary disease (COPD), COPD with AATD, chronic bronchitis, emphysema, and refractory asthma.

19. The method according to claim 16, wherein the disease is COPD.

20. The method according to claim 16, wherein the marker is selected from the group consisting of desmosine, isodesmosine, and combinations thereof.

21. The method according to claim 16, wherein both desmosine and isodesmosine are measured.

22. The method according to claim 16, wherein the candidate compound is selected from the group consisting of hyaluronic acid, polysaccharide, carbohydrate, small molecules, and RNAi.

23. A method for identifying candidate compounds that are effective to treat, prevent, or ameliorate the effects of a disease characterized by elastin degradation comprising:
(a) administering a candidate compound to a cell culture model of the disease;
(b) measuring, by mass spectrometry, the amount of desmosine and isodesmosine in the cell culture administered the candidate compound; and
(c) determining whether the amount of desmosine and isodesmosine produced by the cell culture administered the candidate compound is different compared to a control cell culture absent the candidate compound, wherein a decrease in the amount of the desmosine and isodesmosine produced by the cell culture administered the candidate compound compared to the control cell culture identifies the candidate compound as effective to treat, prevent, or ameliorate the effects of the disease.
